(19) United States
(12) Patent Application Publication
(10) Pub. No.: US 2026/0209680 A1
(43) Pub. Date: Jul. 23, 2026

(54) REAL-TIME, DYNAMIC, BIOREACTOR PROCESS CONTROL USING DYNAMIC METABOLIC FLUX ANALYSIS

(71) Applicant: Metalytics, Inc., Cary, NC (US)

(72) Inventors: Andy Wiranata Wijaya, Crissier (CH); Samuel Yenne, Raleigh, NC (US); Thomas DiProspero, Durham, NC (US); Daniel Isaac Benjamin, San Jose, CA (US); Eric Cumming, Durham, NC (US)

(73) Assignee: Metalytics, Inc., Cary, NC (US)

(21) Appl. No.: 19/091,595

(22) Filed: Mar. 26, 2025

Related U.S. Application Data
(60) Provisional application No. 63/555,529, filed on Feb. 20, 2024.

(51) Int. Cl. C12M 1/36; C12M 1/00; C12M 1/34

(52) U.S. Cl. CPC C12M 41/48; C12M 29/06; C12M 41/46

(57) ABSTRACT

Bioreactor processes have both steady-state and non-steady state sequences from the beginning to the end of the process. Currently, metabolic flux analysis does not provide accurate results for any of the sequences other than steady-state. In order to have a near-real time operation where you are sampling process results, and making changes during a process, you must be able to accurately model both steady-state and non-steady state process sequences. And, you have to do so, quickly. The invention relies upon combining three different modeling techniques to provide accurate results of steady-state and non-steady state process sequences. This capability enables sampling and making changes to bioreactor conditions (temperature, pH, media composition, and so on.). The invention also obviates the need for using isotope traces at all times when modeling the process.

The invention seeks priority with the U.S. provisional application 63/555,529, filed on Feb. 20, 2024.

### TECHNICAL FIELD

The invention is a system and method for dynamic control of bioreactor processes.

### BACKGROUND OF INVENTION

The field of bioprocess engineering has seen significant advancements in recent years, with a growing emphasis on optimizing bioreactor culture processes. Efficient and accurate prediction and forecasting of these processes are critical for ensuring the production of bio-based products, such as drugs, biomaterials, nutritional compounds, and food products.

Bioreactor culture processes involve the cultivation of microorganisms, such as bacteria, yeast, or mammalian cells, for the production of valuable biological products. Accurate predictions and forecasts of these processes are essential for process optimization, cost reduction, and product quality improvement.

Predominantly, at present, bioreactor process management is batch oriented lasting several hours to a few weeks. That is, analysis and process algorithmic changes are done after a batch has been processed using at-line or offline analytics. The new recipe then guides the next batch in the process, and so on. In addition to the time consumed, suboptimal process results can occur that affect end-product quality and process cost. More recently, perfusion bioreactors provide a continuous cell culture process where fresh nutrients are constantly supplied to the bioreactor while simultaneously removing waste products, spent medium, and dead cells. This method maintains a stable environment for cell growth and productivity, enabling high cell densities and extended viability over time. As with batched processes, perfusion bioreactors have variables which can be modified during operation for improved results,

Most agree that if these processes could be analyzed and modified during their operation, this would save time, provide higher quality results, and lower costs over time. The problem is that in order to do so, the analysis has to be able to accurately measure multiple process parameters, feed the measurements into models that can provide quick process profiles, then make dynamic changes to one or more process variables so as to pull the process and its results to an optimal-process state. However contemporary metabolic flux analysis is oriented to steady-state conditions and is notably inaccurate where transitions take place.

### BRIEF DESCRIPTION OF THE INVENTION

The invention herein disclosed is a system and method operative to work in conjunction with a bioreactor (batch or perfusion) system to provide the means for dynamic process control rather than relying on after-the-fact batch process modifications.

In essence, the objective is to forecast metabolic profiles in the cell culture during bioreactor processes. Currently, metabolic flux analysis, whereby the viable cell density is measured against time, is only accurately modeled for steady-state process portions, and cannot be relied upon for dynamic process control.

Looking at a typical metabolic flux analysis graph, one sees that there's a relatively steady state flux density from the beginning of the process called the “lag, followed by a steady increase in viable cell density during the “growth” sequence. Then, there is a protracted steady-state density, again, during the “stationary” sequence, followed by a downturn in density as mortality kicks in. Metabolic flux analysis made during steady-state process sequences may be accurate, but those applied to non-steady-state process sequences could be significantly flawed.

The invention is a system and method for accurately forecasting changing metabolic profiles and the consequent effect upon process adjustments during a process.

The system comprises adjuncts to a bioreactor system that can dynamically gauge metabolic flux and provide near-real time dynamic control of an ongoing process. In addition to a dynamic metabolic model, the invention comprises a molecular-scale model and enzyme kinetic metabolic model.

### BRIEF DESCRIPTION OF DRAWINGS

FIG. 1 depicts an exemplary bioreactor and its components.

FIG. 2 depicts an exemplary batch-oriented analysis and modeling system used in conjunction with the bioreactor of FIG. 1.

FIG. 3 shows an exemplary metabolic flux analysis graph.

FIG. 4 an embodiment of the invention using isotope tracers,

FIG. 5 adds a numerical analysis of historic data to produce a curated enzyme kinetic metabolic model.

FIG. 6 uses the curated model of FIG. 5 for a second embodiment of the invention using no isotope tracers,

FIG. 7 is a process flow of showing the steps related to the embodiment of FIG. 1.

FIG. 8 is a process flow showing the steps related to the embodiment of FIG. 6.

### DETAILED DESCRIPTION OF INVENTION

Bioreactors (batch and perfusion) are precisely controlled environments in which bacteria, yeast, or mammalian cells are cultured to produce biological products, such as drugs, biomaterials, nutritional compounds, and food products.

In order to achieve consistent end results a bioreactor provides precisely controlled parameters such as the rate at which a medium is added, the temperature, air flow, and agitation all while using probes and monitoring subsystems to maintain growth conditions to maximize productivity.

Nearly all such bioreactors are controlled by a predetermined algorithmic control program and produce reasonable consistency of end results.

However, when end results are analyzed and modeled, there is often an opportunity to improve the quality or cost-efficiency of a subsequent batch. Thus, the recipe may be modified, and a new batch is processed. This produce/analyze/model/adjust method leaves room for improvement because it inherently requires after-process adjustments.

One area of a method that can be time consuming is the analysis. It is important that it be accurate and resolute enough to provide meaningful modeling and profiling. Offline measurement, where samples are collected from the process and transported to a remote laboratory for analysis, may allow for more detailed and precise analysis under controlled laboratory conditions, but it is slower and cannot be integrated with real-time production needs.

With in-line analytics, the analyzer is directly integrated into the process line, allowing continuous monitoring of materials as they flow through the system.

At-line analytics, a middle ground between offline and in-line, involves taking a sample from the process and analyzing it near the production line, but outside the actual process stream. It can provide flexibility but is not real time as it involves a delay between sampling and results.

Initially, with at-line and offline processes, samples can be infused with isotope tracers and precisely analyzed. The analysis may point to process adjustments prior to running a subsequent process. That change, however, does not alter results of the previous process.

The invention, herein disclosed, distinguishes itself from contemporary solutions by using dynamic metabolic flux analysis that overcomes the limitations of traditional steady-state metabolic flux analysis approaches. Traditional metabolic flux analysis requires metabolic steady-state conditions. This innovation enables analysis of non-steady state metabolic systems allowing for real-time monitoring and forecasting of changing metabolic states.

The Invention system and method integrates multiple elements comprising: using labeled metabolites from spent media via at-line, in-line, or online analytics; making use of dynamic 13C flux analysis that handles time-varying metabolism extraction of kinetic parameters from this dynamic analysis; and, the combination of kinetic modeling with historical data to forecast cell culture profiles.

Consequently this invention operates without requiring metabolic steady state; handles dynamic changes in metabolic fluxes over time; provides predictive capabilities for cell culture behavior; is adaptable to any culture system and method; and reduces analysis time.

Embodiments of this invention provide a method based on data generated, by any or all of the prior embodiments, for generating dynamic flux rates. This method simulates the dynamic flux rates using temporally resolved, limited experimental data that may or may not utilize an isotopic tracer, and a curated kinetic model derived from a larger kinetic model.

The method, based on data from one or more of other embodiments, improves the flexibility in experimental design, range of uses, and acceptable analytical techniques required to collect experimental data. Furthermore, the subordinate method enables dynamic flux simulations from historical and current experiments and enables applications including, but not limited to, media optimization, culture process optimization, and cell engineering.

In yet a further embodiment of the invention, it provides a method based on data generated by any or all of the prior embodiments for generating dynamic flux rates utilizing isotope tracers. The method provides a curated kinetic model, that is either a subset, matched pair, or extension of the kinetic model, that utilizes the results from previous cultures with isotope tracers to create a curated kinetic model. The curated kinetic model only needs a subset of the extracellular measurements as the enzymatic flux, enzymatic characteristics, and additional model constraints are derived from the outputs of the isotopically labeled model.

This method calculates rates, including but not limited to, external flux rates and concentrations, and simulates internal kinetic fluxes. This curated model requires training, and tests data sets of dynamic flux data derived from experiments with isotopically labeled experiments. The training datasets are used to set constraints of the model, either manually, or with the use of artificial intelligence/machine learning, to define the solution space of the model.

Algorithmically, this invention comprises: automated sequencing batch reactors with time step control; continual sensitivity analysis for non-steady state; and B-splines analysis.

For example, B-spline interpolation is a powerful method for analyzing time-series metabolic data due to its ability to model complex, noisy biological signals while maintaining smoothness and flexibility. B-splines use piecewise polynomials (linear, quadratic, or cubic) to interpolate between data points, avoiding overfitting and unrealistic oscillations common in high-degree polynomials. This is critical for metabolic datasets, where measurement noise can obscure true trends. Metabolic systems are rarely at steady state. B-splines capture time-varying rates (e.g., substrate uptake) by: computing first and second derivatives of interpolated curves to infer metabolic functions (e.g., reaction velocities); and identifying regulatory perturbations (e.g. enzyme activity changes) through derivative ratios.

In one embodiment of the invention, isotope tracers are added in precisely controlled quantity and timing to process samples during the process. Specially engineered dynamic metabolic flux analysis (DMFA) metabolic models joined by molecular-scale models and enzyme kinetic metabolic models provide inputs to a near-real-time process controller subsystem that supports dynamic process control.

As more and more labeled data is gathered from subsequent processes, these may be numerically analyzed to create a curated enzyme kinetic metabolic model. Ordinarily the algorithm could be subject to hundreds or even thousands of variables, both dependent and independent, but the numerical analysis of the historic data enables the curated model to be built that is accurate based on far fewer selected variables and supports dynamic outcomes and forecasting.

For use in future processes, the curated enzyme kinetic metabolic model may use effluent process samples, without need of isotope tracers, to provide the same level of reliable process control as when based on isotope tracer infused samples. This would be a second embodiment of the invention system and method.

The following figures and descriptions are meant to be exemplary and describe two embodiments of the invention system and method. These should not be read as limiting the claims to those specific examples.

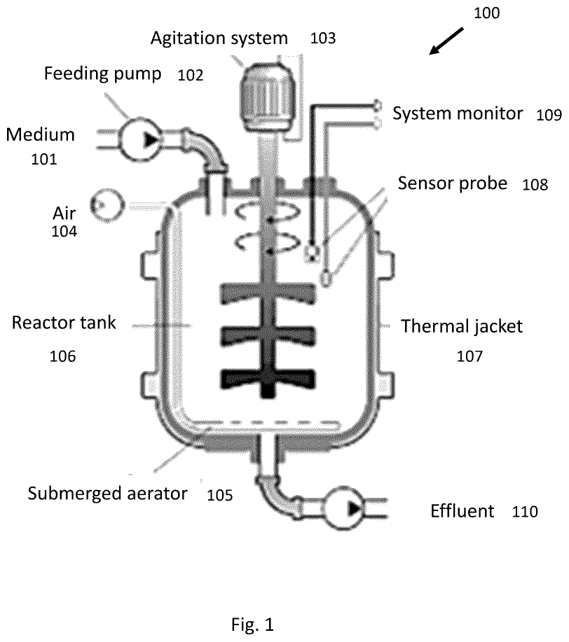

FIG. 1 illustrates a bioreactor (100) comprising a means of introducing a medium (101), feeding the medium into the reactor tank (106) at a controlled rate using a feeding pump (102), stirring the mix with a precisely controlled agitation system (103), aerating the mix with an air input (104) and submerged aerator (105), precisely controlling the temperature using a thermal jacket (107), sensing bioreactor parameters with a sensor probe (108), feeding the probe output to an external system monitor (109) and precisely removing process effluent with a pump (110). As shown, the bioreactor is set up to perform a predetermined recipe while monitoring its parameters.

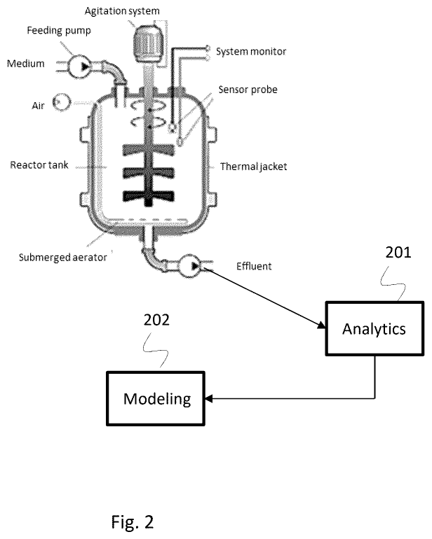

In FIG. 2, the bioreactor of FIG. 1 has an analytic subsystem (201) fed by effluent samples and the analytic results are fed to a modeling subsystem (202). This system adds the capability to that of FIG. 1's system to do analysis, modeling, and process-change on a batched basis.

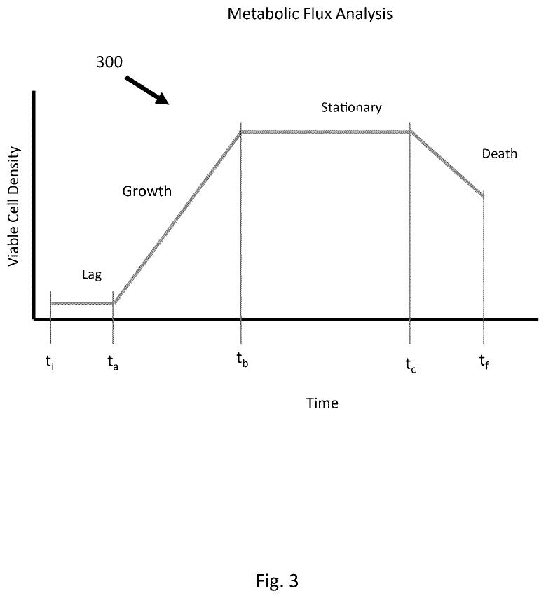

In FIG. 3, as shown, when viable cell density is measured against time, this is called a “metabolic flux analysis.” The two steady-state periods are what is known as the lag which is tᵢ to tₐ, and stationary, which is t_{b} to t_{c}. During the growth sequence, tₐ to t_{b}, viable cell density increases. During the death sequence, t_{c} to t_{f.,} the density decreases. Contemporary systems and methods are unable to accurately map the transitions. As such an analysis done during tᵢ through t_{f} will end up being a best-fit curve that does not accurately depict what's taking place during transitions and consequently would not reliably provide improved process modifications.

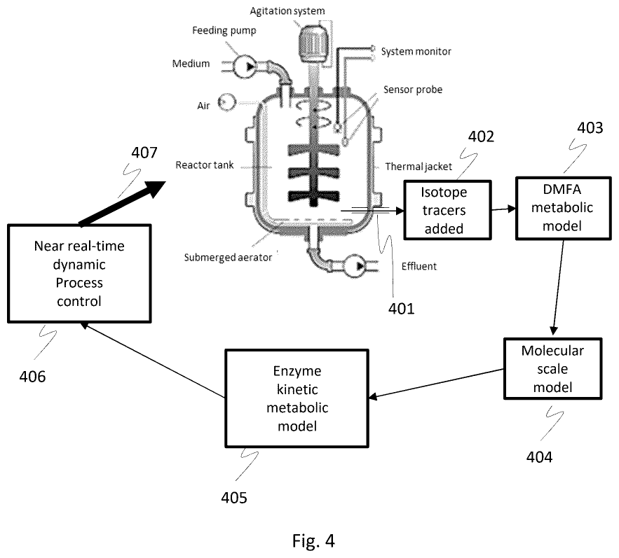

FIG. 4 shows system adjuncts that would be added to the bioreactor of FIG. 1 FIGS. 4, 401, is a slipstream egress that allows a sample of the process at any point in time to have isotope tracers added (402). It is then processed using a dynamic MFA metabolic model, (403), a molecular scale model (404) and an enzyme kinetic metabolic model (405) to provide the input needed by a near real-time dynamic process control subsystem (406) to control the variables of the bioreactor process.

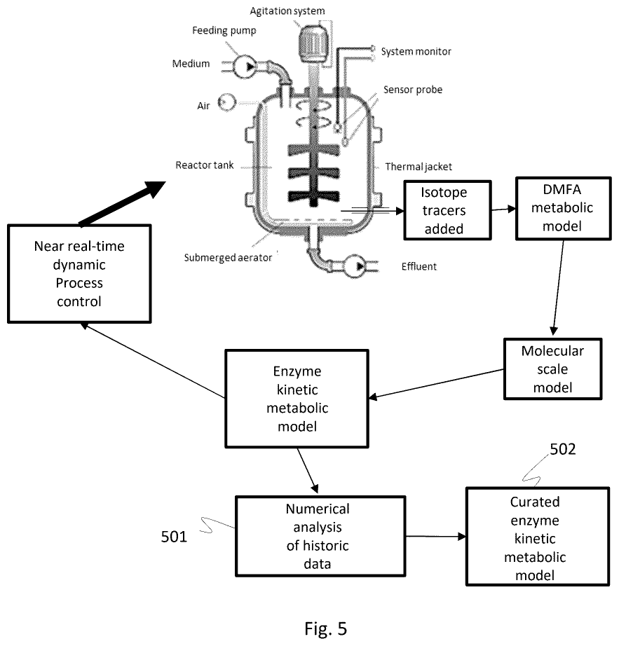

In FIG. 5, the embodiment of FIG. 4 now has a numerical analysis of historic data process (501) producing a curated enzyme kinetic metabolic model (502),

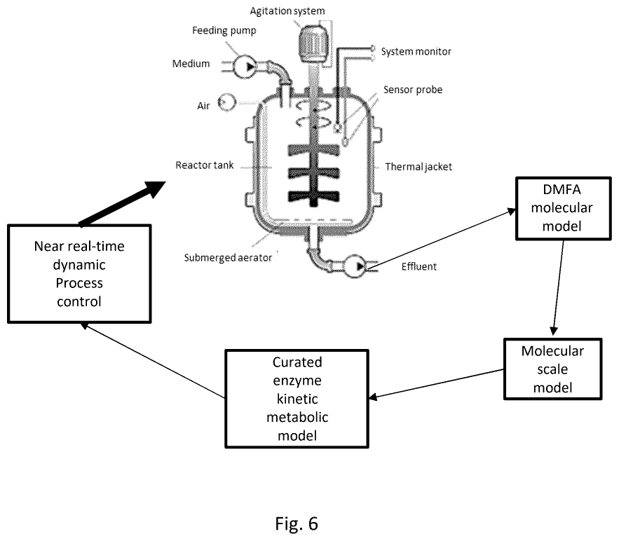

In a second embodiment, FIG. 6, the curated enzyme kinetic metabolic model is then used and it's no longer necessary to use isotope tracers. Both embodiments can provide input to the near real-time dynamic process control subsystem allowing modifications of the process during the process.

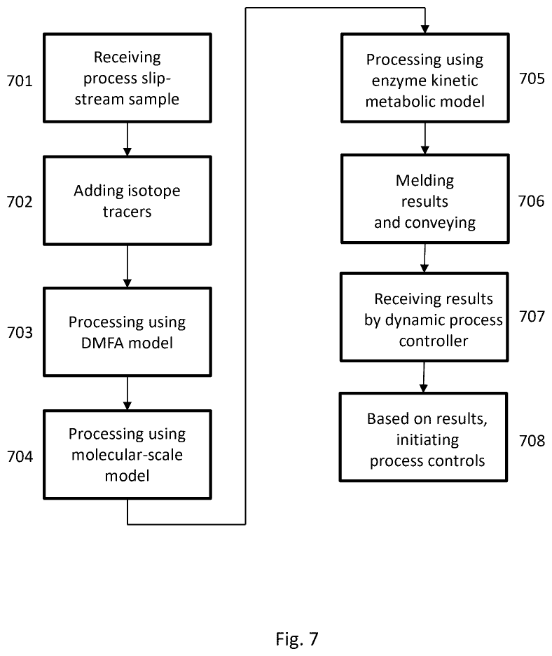

The first embodiment of a method of use is illustrated in FIG. 7. This is based on the system embodiment illustrated in FIG. 4. At any point in time, during the process, a sample is collected from the slipstream (701), and to that sample is added some isotope tracers (702). The infused sample is then processed using the DMFA model (703). Further processing takes place using a molecular scale model (704). And, a final process is done using the enzyme kinetic metabolic model (705). The results are melded and conveyed to the dynamic process controller. The dynamic process control receives those results and based on those results initiates near real-time process controls (708).

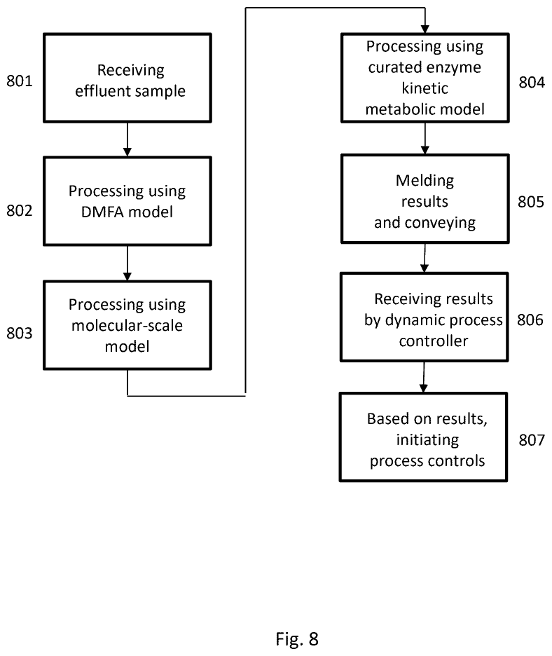

The second embodiment of a method of use is illustrated in FIG. 8. This is based on the system embodiment Illustrated in FIG. 6. At any point in time, during the process, a sample is collected from the effluent exit port (801). That sample is then processed using the DMFA model (802). Further processing takes place using a molecular scale model (803). And a final process is done using the curated enzyme kinetic metabolic model (804). The results are melded and conveyed to the dynamic process controller subsystem (805). Those results are received by the dynamic process controller (806), and based on those results, it then initiates process controls (807).

What is claimed is:

1. A method comprising:
receiving from a bioreactor slipstream a process sample;
infusing the sample with selected isotopic trace elements;
processing an infused sample using dynamic metabolic flux analysis modeling resulting in infused sample data;
processing the infused sample data with a molecular scale model;
processing the infused sample data with an enzyme kinetic metabolic model;
melding the results of the dynamic metabolic flux analysis model with those of the molecular scale model, and those of the enzyme kinetic metabolic model;
conveying melded results to a dynamic process controller; and
initiating process controls based on the melded results.

2. A method comprising:
receiving from a bioreactor an effluent process sample;
processing the effluent process sample using the dynamic metabolic flux analysis modeling;
processing the results of the dynamic metabolic flux analysis modeling with the molecular scale model;
processing the results of the dynamic metabolic flux analysis modeling and the molecular scale modeling with a curated enzyme kinetic metabolic model;
melding the results of the dynamic metabolic flux analysis model with those of the molecular scale model, and those of the curated enzyme kinetic metabolic model;
conveying melded results to the dynamic process controller; and
initiating process controls based on the melded results.

3. A system comprising:
a dynamic metabolic flux analysis modeling subsystem operative to accurately model both steady-state and non-steady state reactor process sequences;
a molecular scale modeling subsystem; and
an enzyme kinetic metabolic model subsystem.

4. The system as in claim 3 further comprising:
a numerical analysis processing subsystem; and
a curated enzyme kinetic metabolic model subsystem.

5. The system as in claim 4 wherein the numerical analysis subsystem operates on historic process data in conjunction with machine-learning to create increasingly accurate model results.

6. The system as in claim 4 wherein the curated enzyme kinetic metabolic model subsystem is operative to apply algorithms to produce accurate metabolic state forecasts at any point within an active process.
